(19) United States
(12) Patent Application Publication
(10) Pub. No.: US 2023/0017867 A1
(43) Pub. Date: Jan. 19, 2023

(54) Systems, Methods, and Media for Automatically Predicting a Classification of Incidental Adrenal Tumors Based on Clinical Variables and Urinary Steroid Levels

(71) Applicant: Mayo Foundation for Medical Education and Research, Rochester, MN (US)

(72) Inventors: Irina Bancos, Rochester, MN (US); Dennis Haaga Murphree, JR., Rochester, MN (US); Eric C. Polley, Rochester, MN (US)

(21) Appl. No.: 17/782,378

(22) PCT Filed: Dec. 7, 2020
(86) PCT No.: PCT/US2020/063626

§ 371 Date: Jun. 3, 2022

Related U.S. Application Data
(60) Provisional application No. 62/944,140, filed on Dec. 5, 2019.

(51) Int. Cl. G16B 40/00; G16H 50/70; G16B 5/20

(52) U.S. Cl. CPC G16B 40/00; G16H 50/70; G16B 5/20

(57) ABSTRACT

In accordance with some embodiments, systems, methods, and media for automatically predicting a classification of incidental adrenal tumors based on clinical variables and urinary steroid levels are provided. In some embodiments, the system comprises: a processor programmed to: generate a feature vector including clinical variables and biomarker levels associated with the patient presenting with an unclassified adrenal mass; provide the feature vector to a machine learning model trained using a labeled feature vectors associated patients having adrenal masses classified as benign, adrenal cortical carcinoma, or another malignant adrenal mass; receive, from the trained machine learning model, an output indicative of a classification of the unclassified adrenal mass; and cause information indicative of the classification to be presented to a user to aid the user in classification of the unclassified adrenal mass.

### CROSS-REFERENCE TO RELATED APPLICATIONS

This application claims the benefit of U.S. Provisional Patent Application No. 62/944,140, filed Dec. 5, 2019, which is hereby incorporated herein by reference in its entirety for all purposes.

### STATEMENT REGARDING FEDERALLY SPONSORED RESEARCH

N/A

### BACKGROUND

Adrenal tumors are serendipitously found in approximately 5% of the tens of millions of computed tomography (CT) scans of the anatomy in the vicinity of the adrenal gland performed in the U.S. each year (note that adrenal masses discovered serendipitously on a radiological scan are sometimes referred to as incidental adrenal tumors or incidental adrenal masses). The prevalence of adrenal masses generally increases with age ranging from less than 0.5% in children and around 10% in 70-year-old patients. Because the number of radiological scans that are performed is also correlated with age, the probability of discovering an incidental adrenal tumor dramatically increases with age. Although the majority of these tumors may be inactive or benign, the survival rate for the malignant tumors is very poor.

Of patients with incidental adrenal masses evaluated in endocrine clinics, 8% are diagnosed with malignant adrenal tumors a majority of which are diagnosed as adrenal cortical carcinomas (ACC). However, other malignancies are also diagnosed such as sarcomas and lymphomas. ACCs are rare tumors that typically have a very aggressive course and high mortality unless diagnosed at an early stage. Unfortunately, CT (the most common imaging modality used in the evaluation of such tumors) is limited in its ability to provide features that can be used to distinguish benign from malignant adrenal tumors. At least one third of all benign tumors demonstrate indeterminate imaging characteristics. Additional diagnostic procedures used to inform diagnosis frequently include further costly imaging using different modalities (e.g., magnetic resonance imaging (MRI)), imaging repeatedly over time to assess for any growth in the tumor, adrenal biopsy and not infrequently, adrenalectomy. This uncertainty can cause patients that in fact had a benign adrenal tumor (e.g., determined based on an evaluation by a pathologist using a tissue sample of the tumor collected during an adrenalectomy) to undergo unnecessary surgery, while some patients with ACC may experience an unacceptable delay in surgery while waiting to see if the adrenal mass grows. As it stands, clinicians must generally rely on their acumen to determine the likelihood of an adrenal tumor being malignant or benign, which is a complex decision that generally is made based on tumor size, imaging characteristics and production of a few steroid hormones that can be routinely tested.

Clinical assessment of probability for malignancy can generate relatively good results when expert physicians are involved and the tumors are relatively large (e.g., relatively high precision in not missing tumors that are malignant). However, results for small and medium sized tumors are more difficult to assess, which often leads to repeat imaging and extensive follow-up, and in some cases surgical exploration is required to arrive at a definitive diagnosis.

Accordingly, systems, methods, and media for automatically predicting a classification of incidental adrenal tumors based on clinical variables and urinary steroid levels are desirable.

### SUMMARY

In accordance with some embodiments of the disclosed subject matter, systems, methods, and media for automatically predicting a classification of incidental adrenal tumors based on clinical variables and urinary steroid levels are provided.

In accordance with some embodiments of the disclosed subject matter, a system for predicting a classification of an adrenal mass is provided, the system comprising: at least one hardware processor that is programmed to: generate a feature vector that includes a first plurality of values and a second plurality of values, wherein the first plurality of values corresponds to a respective plurality of clinical variables associated with a patient presenting with an unclassified adrenal mass, and the second plurality of values corresponds to a respective plurality of biomarker levels associated with the patient presenting with the unclassified adrenal mass; provide the feature vector to a trained machine learning model, wherein the machine learning model was trained using a plurality of labeled feature vectors associated with a respective plurality of patients having a classified adrenal mass, wherein each of the plurality of feature vectors included values corresponding to the plurality of clinical variables and the plurality of biomarker levels associated with a respective patient, and each of the plurality of feature vectors is associated with an indication of a diagnosis of the respective classified adrenal mass as being one of benign, adrenal cortical carcinoma (ACC), and a malignant adrenal mass other than ACC; receive, from the trained machine learning model, an output indicative of a classification of the unclassified adrenal mass; and cause information indicative of the classification to be presented to a user to aid the user in classification of the unclassified adrenal mass.

In some embodiments, the trained machine learning model is a gradient boosting machine model comprising a plurality of decision trees.

In some embodiments, the plurality of clinical variables includes an unenhanced Hounsfield unit value of the adrenal mass, a size of the adrenal mass, and an indication of whether the patient was experiencing an excess of hormones excreted by the adrenal gland.

In some embodiments, the plurality of biomarker levels includes at least ten levels of biomarkers indicative of at least one of a steroid, a steroid precursor, and a metabolite that falls within the mineralocorticoid, glucocorticoid, or androgen pathways of adrenal steroidogenesis extracted from a 24-hour urine sample.

In some embodiments, the output comprises a plurality of values each indicative of a likelihood that the unclassified adrenal mass is a member of each class of adrenal mass, wherein the classes of adrenal mass comprise benign, ACC, and malignant adrenal mass other than ACC.

In some embodiments, the system further comprises a liquid chromatography high-resolution accurate-mass (LC-HRAM) spectrometer, and the at least one hardware processor that is further programmed to: receive a plurality of biomarker levels from the LC-HRAM spectrometer; and generate the second plurality of values using the plurality of biomarker levels.

In some embodiments, the second plurality of values comprises a plurality of z-scores each indicative of a level of a particular biomarker.

In some embodiments, the plurality of biomarkers correspond to at least twenty of the following: 6B-hydroxycortisol, Cortisol, Cortisone, B-Cortolone, a-cortolone, 16a-Dephdroepi-androsterone, 5a-Tetrahydrocortisol, Tetrahydrocortisol, Tetrahydrocortisone, Pregnanteriolone, Tetrahydrocorticosterone, 11-Oxo-etiocholanolone, 5-Pregnanetriol, 11B-Hydroxy-etiocholanolone, Tetrahydro-11-deoxycortisol, Dehdroepiandrosterone, Pregnanetriol, Tetrahydrodeoxy-corticosterone, 5-Pregnenediol, 5a-Tetra-11-dehydrocotricosterone, Etiocholanolone, Androsterone, 17-OH-pregnanolone, and Pregnanediol.

In some embodiments, the at least one hardware processor that is further programmed to: receive the plurality of clinical variables from an electronic medical record system; and generate the first plurality of values using the plurality of clinical variables.

In accordance with some embodiments of the disclosed subject matter, a method for predicting a classification of an adrenal mass is provided, the method comprising: generating a feature vector that includes a first plurality of values and a second plurality of values, wherein the first plurality of values corresponds to a respective plurality of clinical variables associated with a patient presenting with an unclassified adrenal mass, and the second plurality of values corresponds to a respective plurality of biomarker levels associated with the patient presenting with the unclassified adrenal mass; providing the feature vector to a trained machine learning model, wherein the machine learning model was trained using a plurality of labeled feature vectors associated with a respective plurality of patients having a classified adrenal mass, wherein each of the plurality of feature vectors included values corresponding to the plurality of clinical variables and the plurality of biomarker levels associated with a respective patient, and each of the plurality of feature vectors is associated with an indication of a diagnosis of the respective classified adrenal mass as being one of benign, adrenal cortical carcinoma (ACC), and a malignant adrenal mass other than ACC; receiving, from the trained machine learning model, an output indicative of a classification of the unclassified adrenal mass; and causing information indicative of the classification to be presented to a user to aid the user in classification of the unclassified adrenal mass.

In accordance with some embodiments of the disclosed subject matter, a non-transitory computer readable medium containing computer executable instructions that, when executed by a processor, cause the processor to perform a method for predicting a classification of an adrenal mass is provided, the method comprising: generating a feature vector that includes a first plurality of values and a second plurality of values, wherein the first plurality of values corresponds to a respective plurality of clinical variables associated with a patient presenting with an unclassified adrenal mass, and the second plurality of values corresponds to a respective plurality of biomarker levels associated with the patient presenting with the unclassified adrenal mass; providing the feature vector to a trained machine learning model, wherein the machine learning model was trained using a plurality of labeled feature vectors associated with a respective plurality of patients having a classified adrenal mass, wherein each of the plurality of feature vectors included values corresponding to the plurality of clinical variables and the plurality of biomarker levels associated with a respective patient, and each of the plurality of feature vectors is associated with an indication of a diagnosis of the respective classified adrenal mass as being one of benign, adrenal cortical carcinoma (ACC), and a malignant adrenal mass other than ACC; receiving, from the trained machine learning model, an output indicative of a classification of the unclassified adrenal mass; and causing information indicative of the classification to be presented to a user to aid the user in classification of the unclassified adrenal mass.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

The patent or application file contains at least one drawing executed in color. Copies of this patent or patent application publication with color drawing(s) will be provided by the Office upon request and payment of the necessary fee.

FIG. 1 shows an example of a system for automatically predicting a classification of incidental adrenal tumors based on clinical variables and urinary steroid levels in accordance with some embodiments of the disclosed subject matter.

FIG. 2 shows an example of hardware that can be used to implement a computing device, and a server, shown in FIG. 1 in accordance with some embodiments of the disclosed subject matter.

FIG. 3 shows an example of a flow for training and using mechanisms for automatically predicting a classification of incidental adrenal tumors based on clinical variables and urinary steroid levels in accordance with some embodiments of the disclosed subject matter.

FIG. 4 shows an example of a process for training a machine learning model for automatically predicting a classification of incidental adrenal tumors based on clinical variables and urinary steroid levels in accordance with some embodiments of the disclosed subject matter.

FIG. 5 shows an example of a process for using a machine learning model for automatically predicting a classification of incidental adrenal tumors based on clinical variables and urinary steroid levels in accordance with some embodiments of the disclosed subject matter.

FIGS. 6A1 to 6A4 show an example of a report that can be generated based on an output of a system for automatically predicting a classification of incidental adrenal tumors based on clinical variables and urinary steroid levels in accordance with some embodiments of the disclosed subject matter.

FIGS. 6B1 to 6B4 show another example of a report that can be generated based on an output of a system for automatically predicting a classification of incidental adrenal tumors based on clinical variables and urinary steroid levels in accordance with some embodiments of the disclosed subject matter.

FIGS. 6C1 to 6C4 show yet another example of a report that can be generated based on an output of a system for automatically predicting a classification of incidental adrenal tumors based on clinical variables and urinary steroid levels in accordance with some embodiments of the disclosed subject matter.

### DETAILED DESCRIPTION

In accordance with various embodiments, mechanisms (which can, for example, include systems, methods, and media) for automatically predicting a classification of incidental adrenal tumors based on clinical variables and urinary steroid levels are provided.

In some embodiments, mechanisms described herein can automatically generate a prediction that is indicative of a classification of an adrenal mass. For example, the mechanisms can predict whether a particular adrenal mass is benign, is an ACC tumor, and/or another type of malignant adrenal tumor. In a more particular example, the mechanisms can provide a likelihood that the adrenal mass is a member of each of the classes.

In some embodiments, mechanisms described herein can use any suitable variables associated with the patient and/or adrenal mass to predict a classification of an adrenal mass, such as one or more variables describing a current and/or past state of the patient presenting with the adrenal tumor, one or more variables describing the circumstances under which the adrenal mass was discovered, and/or one or more variables describing the current and/or past state of the adrenal mass. For example, variables describing a current and/or past state of the patient presenting with the adrenal mass can include an age of the patient when the adrenal mass was discovered, sex of the patient, whether the patient is experiencing adrenal hyperfunction, and/or the presence and/or level of one or more analytes in a fluid sample collected from the patient (e.g., the level of one or more steroids in a sample of the patient's urine) which are sometimes referred to herein as biomarkers. In a particular example, adrenal hormone hyperfunction can be determined based on standard of care tests, including 1 milligram (mg) dexamethasone suppression, measurements of plasma aldosterone and renin concentrations, and 24-hour urine measurements of cortisol.

As another example, a variable describing the circumstances under which the adrenal mass was discovered can include whether the adrenal mass was discovered incidentally (e.g., the mass was discovered in a CT scan that was ordered for another reason), intentionally (e.g., the mass was discovered in a CT scan that was ordered to determine whether an adrenal mass was present—for example, as a part of cancer staging imaging for a known extra-adrenal malignancy, or to investigate the source of adrenal hormonal excess such as Cushing syndrome, hypertension associated with low potassium, etc.), or another way.

As yet another example, variables describing the current and/or past state of the adrenal mass can include the size of the adrenal mass (e.g., based on the largest tumor diameter measurement),measurement) and/or an unenhanced Hounsfield unit measurement associated with the adrenal mass in a CT scan. In a particular example, Hounsfield unit measurement cancan be an actual Hounsfield unit take from an unenhanced CT scan showing a homogeneous lesion. If a CT scan shows a heterogeneous lesion, Hounsfield unit measurement can be defined in an indeterminate range (e.g., >20), and cancan be recorded as such.

In still another example, the variables used by mechanisms described herein can include clinical variables such as: age at diagnosis; sex; tumor size; Unenhanced Hounsfield unit measurement on CT; mode of discovery; presence/absence of adrenal hyperfunction. These data are generally readily available for most patients with an adrenal mass and can be used alone to calculate a pre-test probability of ACC, other malignant mass, and benign adrenal mass with 95% accuracy to diagnose a malignant mass (including ACC and other malignancies), but less accuracy to distinguish ACC from other malignant tumors. In such an example, levels of various steroids can be profiled based on a urine assay performed using one or more liquid chromatography high-resolution accurate-mass (LC-HRAM) spectrometry techniques can be used as additional variables. In such an example, the steroid profiling can be used to quantify over twenty steroids, steroid precursors and metabolites within the mineralocorticoid, glucocorticoid and androgen pathways of adrenal steroidogenesis in a 24-hour urine sample. Liquid chromatographic separation coupled with the high resolution capabilities of an HRAM device such as a Q-Exactive Hybrid QuadrupoleQuadrupole Orbitrap™ mass spectrometer available from ThermoFisher Scientific, which can allow for unequivocal identification of all 20+ steroids while maintaining a high throughput workflow. Steroid profiling alone can provide an accuracy for diagnosing ACC on the order of 90-95%, and when combined with clinical variables described above can facilitate an accurate, rapid and cost-effective diagnosis or post-test prediction of ACC, other malignancy, and benign adrenal masses. Human adrenal glands produce three types of steroid hormones: mineralocorticoids, glucocorticoids, and sex steroids, which are all derived from cholesterol via several intermediate steps. Benign adrenal adenomas (AAs) produce similar steroid in proportions that are similar to that produced in normal adrenal tissue, with near-normal levels of precursor- and bioactive steroids being produced. By contrast, ACC frequently exhibit abnormal patterns of steroid production. By measuring 20+ different steroid metabolites, even subtle abnormalities can be detected and ACCs can be distinguished from AAs.

In some embodiments, mechanisms described herein can use any suitable variables associated with the patient and/or adrenal mass to train one or more machine learning models to predict a classification of an adrenal mass based on similar variables. In some embodiments, mechanisms described herein can train any suitable type of machine learning model or models to predict a classification of an adrenal mass. For example, mechanisms described herein can train a gradient boosting machine (GBM) based on simple decision trees using sets of variables associated with a particular patient and with a label indicating the class of adrenal mass (e.g., benign, ACC, or other malignant adrenal mass). As another example, mechanisms described herein can train a model using penalized multinomial logistic regression techniques using sets of variables associated with a particular patient (e.g., variables described herein in connection with GBM-based models) and with a label indicating the class of adrenal mass (e.g., benign, ACC, or other malignant adrenal mass). As yet another example, mechanisms described herein can train a model using penalized elastic net regression techniques using sets of variables associated with a particular patient (e.g., variables described herein in connection with GBM-based models) and with a label indicating the class of adrenal mass (e.g., benign, ACC, or other malignant adrenal mass). As still another example, mechanisms described herein can train a model using least absolute shrinkage and selection operator (LASSO) regression techniques using sets of variables associated with a particular patient (e.g., variables described herein in connection with GBM-based models) and with a label indicating the class of adrenal mass (e.g., benign, ACC, or other malignant adrenal mass). As a further example, mechanisms described herein can train a model using ridge regression techniques using sets of variables associated with a particular patient (e.g., variables described herein in connection with GBM-based models) and with a label indicating the class of adrenal mass (e.g., benign, ACC, or other malignant adrenal mass).

In some embodiments, mechanisms described herein can train a machine learning model to minimize the risk of false negatives (i.e., identifying a malignant tumor as benign), to minimize the risk of false positives (i.e., identifying a benign mass as an ACC or other malignancy), or to provide a relatively balanced tradeoff between false negatives and false positives. In general, tree-based models are a form of statistical learning that can capture non-linear relationships between independent variables that are included, whereas commonly used linear models such as binomial or multinomial logistic regression generally are not able to capture such non-linear relationships. Tree-based models can be characterized as a set of if-then statements that are constructed based on training data that can be applied to new data to make a prediction. For example, an optimal set of such if-then statements can be constructed by choosing those that minimize prediction errors on the training data. Additionally, GBM techniques are generally more robust to missing data (e.g., a missing data point in a feature vector for a particular patient), and implicitly considers interactions, as well as being less sensitive to predictor variable correlation and scale than other types of tree-based model.

More generally, tree-based models can be used in a boosting framework in which a series of new trees is sequentially fit to modified versions of the training data. Such combination of many weak models (e.g., simple decision trees) into a more complex ensemble can overcome many of the limitations of models that use only a single tree. An example boosting framework assigns weights to the observations in the training data after training a first tree in the sequence, with misclassified observations receiving higher weights and correctly classified observations receiving lower weights. A subsequent tree can then be trained on the weighted dataset and new weights can subsequently be assigned based on performance. In such a boosting framework, the final sequence of trees, often called an ensemble, can be used to produce predictions based on the weighted sum of its constituent trees. As another example, sequential re-weighting of training observations based on the error can be omitted, and new trees can instead be trained directly on the prediction errors made by previous trees, which are sometimes referred to as residuals. An initial tree in the sequence can predict the outcome of interest (e.g., the category of an adrenal mass), and each new tree that is added to the model can be trained on the prediction errors from the previous model, and a new tree which maximizes the reduction in error can be added to the previous sequence of trees to form a new model. This sequence can be repeated until an appropriate level of error is achieved or another stopping condition is met.

In some embodiments, mechanisms described herein can use one or more trained machine learning models to determine a likely classification of an adrenal mass, and use the output to present information to a user (e.g., a medical professional such as an oncologist), for example, in the form of a report. In such embodiments, the user can evaluate the output produced by the machine learning model(s) to determine a recommend course of treatment and/or additional evaluations to recommend, if any.

In some embodiments, mechanisms described herein can facilitate diagnosis of adrenal masses that is more accurate when compared to conventional diagnostic procedures at a lower cost, with less reliance on invasive procedures that can cause patient's harm, and/or with less radiation exposure. A result generated using mechanisms described herein can provide a referring physician a highly accurate probability that can facilitate selection of a more optimal clinical path forward based on an informed discussion between physician and patient. For example, using mechanisms described herein that predict a classification of an adrenal mass based on clinical variables and biomarkers, a diagnosis can be made more quickly on relatively small indeterminate tumors that are not susceptible to accurate diagnosis based on radiology images alone (e.g., based on a CT scan). This can help avoid unnecessary follow-up imaging visits, unneeded biopsies, or even adrenalectomy (i.e., where the entire mass is removed to reach a diagnosis), especially when the prediction generated by the mechanism is a robust likelihood that the adrenal mass is benign, which can avoid substantial health care costs, patient anxiety, and the potential for patient harm as a side effect of unnecessary diagnostic tests or treatments. In such an example, patients that are diagnosed with a small ACC using mechanisms described herein can lead to earlier intervention that has the potential to radically improve patient prognoses compared to treatment when ACC diagnosis has been confirmed using conventional techniques that rely on follow up imaging and/or eventual biopsy.

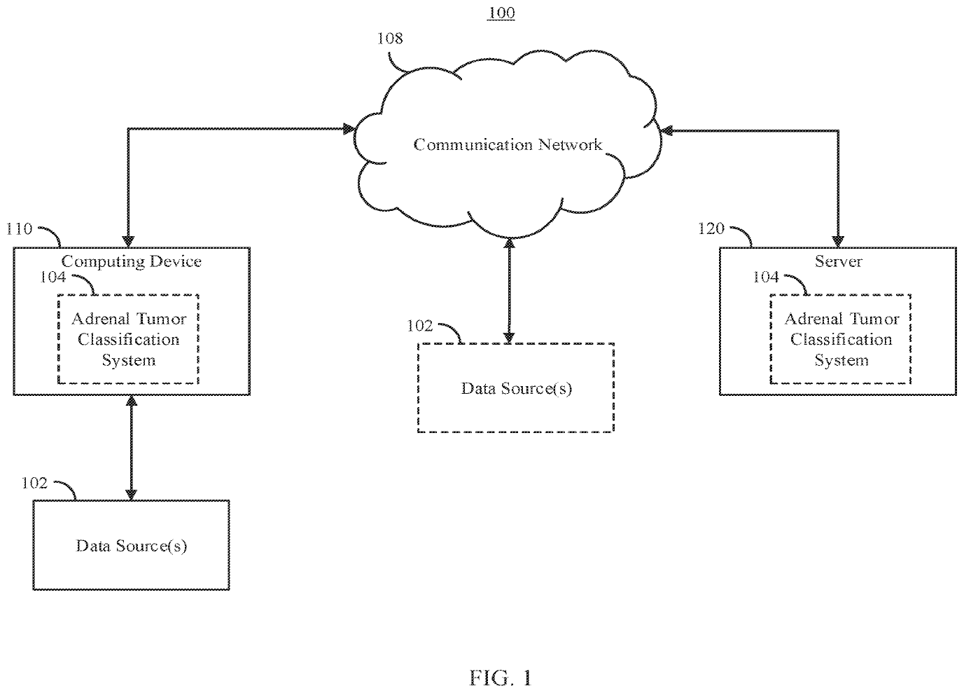

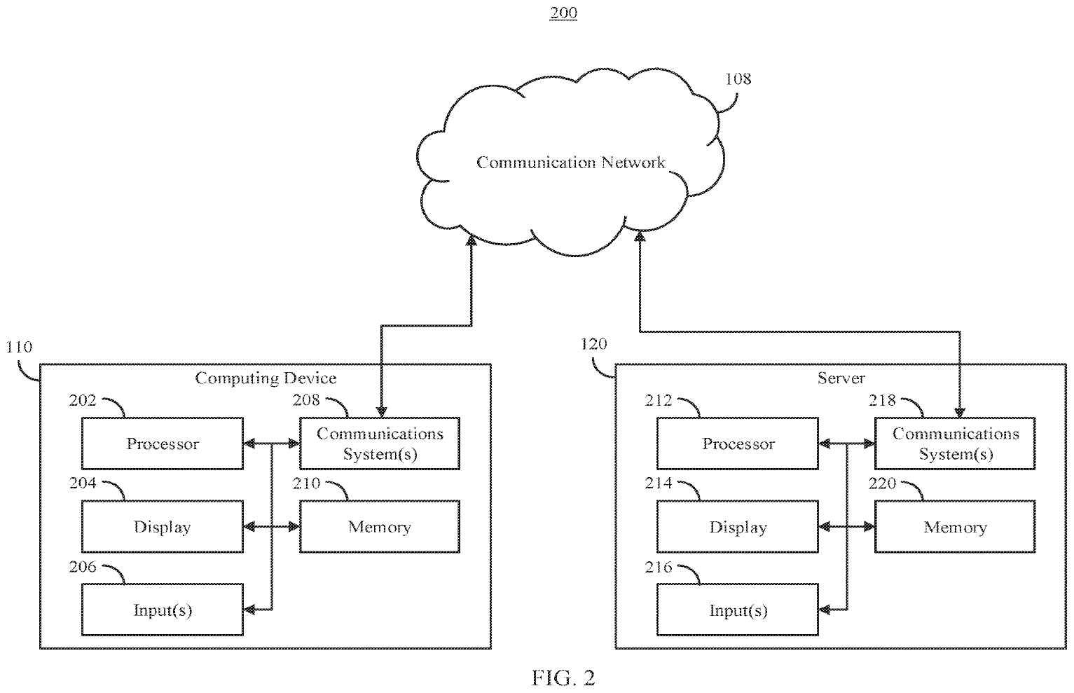

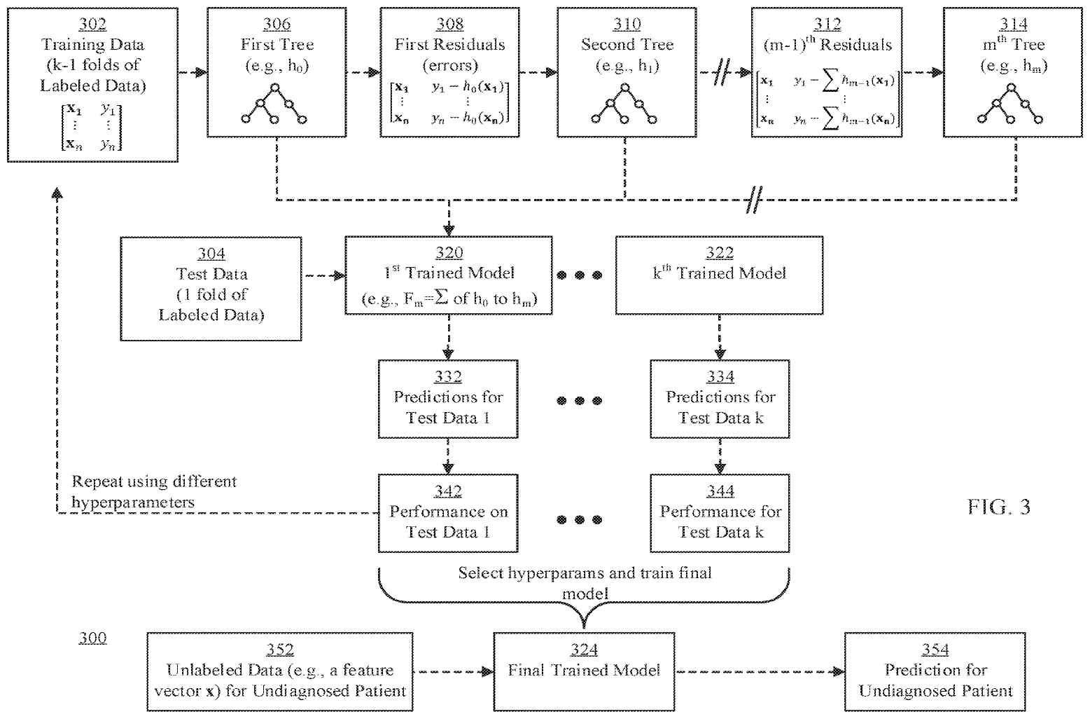

FIG. 1 shows an example 100 of a system for predicting a classification of incidental adrenal tumors based on clinical variables and urinary steroid levels in accordance with some embodiments of the disclosed subject matter. As shown in FIG. 1, a computing device 110 can receive clinical variables and/or steroid levels from a data source 102 that stores such data. In some embodiments, computing device 110 can execute at least a portion of an adrenal tumor classification system 104 to automatically predict a classification of incidental adrenal tumors based on clinical variables and urinary steroid levels.

Additionally or alternatively, in some embodiments, computing device 110 can communicate information about clinical variables and/or steroid levels from data source 102 to a server 120 over a communication network 108 and/or server 120 can receive clinical variables and/or steroid levels from data source 102 (e.g., directly and/or using communication network 108), which can execute at least a portion of adrenal tumor classification system 104 to automatically predict a classification of incidental adrenal tumors based on clinical variables and urinary steroid levels. In such embodiments, server 120 can return information to computing device 110 (and/or any other suitable computing device) indicative of a predicted classification of the incidental adrenal tumors.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, etc. As described below in connection with FIGS. 3-5, in some embodiments, computing device 110 and/or server 120 can receive labeled data (e.g., clinical variables and steroid levels) from one or more data sources (e.g., data source 102), and can format the clinical variables and/or steroid levels for use in training a machine learning model to be used to provide adrenal tumor classification system 104. In some embodiments, adrenal tumor classification system 104 can use the labeled data to train a machine learning model(s) to classify adrenal tumors using unlabeled data from a patient presenting with an adrenal mass that has not yet been diagnosed with sufficient confidence. For example, the steroid levels can be steroid excretion levels generated techniques to assay a urine sample, and each of the steroid excretion values can be log-transformed and subsequently z-score normalized with respect to the mean and standard deviation associated with each steroid in the data set.

In some embodiments, adrenal tumor classification system 104 can receive unlabeled data (e.g., clinical variables and steroid levels) from one or more sources of data (e.g., data source 102), and can format the clinical variables and/or steroid levels for input to the trained machine learning model(s). In some embodiments, adrenal tumor classification system 104 can generate a predicted classification of the adrenal mass, and can present the results for a user (e.g., a physician, a nurse, a paramedic, etc.).

In some embodiments, data source 102 can be any suitable source or sources of clinical variables and/or steroid levels. For example, data source 102 can be an electronic medical records system. As another example, data source 102 can be an LC-HRAM spectrometer. As yet another example, data source 102 can be an input device that facilitates manual data entry by a user. As still another example, data source 102 can be data stored in memory of computing device 110 and/or server 120 using any suitable format, such as using a database, a spreadsheet, a document with data entered using a comma separated value (CSV format), and/or any other suitable format.

In some embodiments, data source 102 can be local to computing device 110. For example, data source 102 can be incorporated with computing device 110 (e.g., using memory associated with computing device). As another example, data source 102 can be connected to computing device 110 by one or more cables, a direct wireless link, etc. Additionally or alternatively, in some embodiments, data source 102 can be located locally and/or remotely from computing device 110, and can data to computing device 110 (and/or server 120) via a communication network (e.g., communication network 108).

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

FIG. 2 shows an example 200 of hardware that can be used to implement computing device 110, and/or server 120 in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 204 can include any suitable display devices, such as a computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to present content (e.g., user interfaces, graphics, tables, reports, etc.), receive content from server 120, transmit information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an MCU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to transmit information and/or content (e.g., a user interface, graphs, tables, reports, etc.) to one or more computing devices 110, receive information and/or content from one or more computing devices 110, receive instructions from one or more devices (e.g., a personal computer, a laptop computer, a tablet computer, a smartphone, etc.), etc.

FIG. 3 shows an example 300 of a flow for training and using mechanisms for automatically predicting a classification of incidental adrenal tumors based on clinical variables and urinary steroid levels in accordance with some embodiments of the disclosed subject matter. As shown in FIG. 3, labeled data can be used to train multiple machine learning models to predict a classification of an adrenal mass. In some embodiments, labeled data can include data sets for various patients for which data was collected at an appropriate point (or points) in time (e.g., at a time when the diagnosis of the adrenal mass was not yet definitively determined), and for which a definitive diagnosis was made (e.g., based on a tissue sample collected via biopsy or adrenalectomy). In some embodiments, the data associated with each patient can include various data points. For example, the data associated with each patient can include one or more clinical variables (e.g., values indicative of age at diagnosis; sex; tumor size; Unenhanced Hounsfield unit measurement on CT; mode of discovery; and/or presence/absence of adrenal hyperfunction) and/or one or more biomarkers (e.g., values indicative levels of various steroids determined via an assay of a urine sample). As another example, the data associated with each patient can include a ground truth diagnosis associated with the patient.

In some embodiments, data associated with each patient can be formatted as a vector x with a length corresponding to the total number of features on which the machine learning model is to be trained, and a value y representing the diagnosis associated with the patient. For example, if the patient data to be used in training includes six clinical variables and 26 biomarker levels, the vector x can have a length of 32 with each position corresponding to a particular variable and having a value indicative of the value of the variable. In some embodiments, the diagnosis for each patient can be coded as a factor having multiple levels, which an integer value corresponding to a particular diagnosis. For example, benign, other malignant, and ACC can be coded as integer values 1, 2, and 3, respectively. As another example, benign, other malignant, and ACC can be coded as integer values −1, 0, and 1, respectively. Note that these are merely examples, and diagnosis can be coded using other schemes. As described above, the biomarker levels can be formatted using any suitable technique or combination of techniques. For example, the biomarkers can be log-transformed and z-score normalized based on the mean and standard deviation for that biomarker in the data set.

In some embodiments, the training data can be grouped into any suitable number of folds that each have a distribution of diagnoses that is similar to the overall distribution of diagnoses. For example, the labeled data can be grouped into five folds that each include a roughly equal number of patients. In a more particular example, the labeled data can include 401 patients, of which 351 were diagnosed with a benign tumor, 29 were diagnosed with an ACC tumor, and 21 were diagnosed with a malignant adrenal tumor that was not an ACC tumor. These 401 patients can be divided into five groups each representing 80 or 81 patients, with about 70 benign, 6 ACC, and 4 other malignancy in each group.

In some embodiments, a set of training data 302 can include all but one of the folds. In general, cross-validation is an approach to training statistical learning models that provides a way of assessing how a model can be expected to generalize to different datasets. For example, if the labeled data has been divided into five folds, training data 302 can include four of the five folds to be used to train a first machine learning model. In such embodiments, a fold (of folds) not included in training data 302 can be used as test data 304, which can be used to evaluate the performance of a trained model. As described above, in such a five-fold cross-validation, the training data can be divided into five equal sections which can be referred to as folds, each of which maintains the same class balance of the dataset as the whole dataset. A model can be trained on four of the five folds and is assessed using the fifth fold. This can be repeated five times using a different assessment fold each time, and the performance of the models on each fold can be compared.

In some embodiments, a grid search can be conducted to determine values for hyperparameters, such as maximum number of trees (m), learning rate (η), shrinkage, and maximum interaction depth. In such embodiments, multiple models can be generated using various combinations of hyperparameter values, and can be evaluated to determine which hyperparameters generate superior performing models. After evaluating the performance of the various models and selecting hyperparameters that produce best results, the final model can be produced by training on all available labeled data.

In some embodiments, training data 302 can be used to generate a first tree 306 using any suitable technique or combination of techniques. For example, first tree 306 can be a simple tree that is generated using training data 302 and one or more hyperparameters, such as a maximum interaction depth that can limit the number of splits (e.g., if-then statements) allowed between the root and the deepest leaf node, that are allowed in each of the constituent trees. In some embodiments, first tree 306 can be automatically generated using any suitable tree generation technique or combination of techniques. For example, first tree 306 can be generated by determining at each node which feature of the remaining features that have not been selected in the current tree can be used to split the patients associated with that node into new nodes that minimize prediction error. This can be done recursively until a stopping condition is reached, such as a minimum number of patients (e.g., one, two, etc.) has been reached, a maximum depth has been reached, or if another division would fail to improve prediction accuracy (e.g., if the current group is homogenous in class, dividing the group again may not provide additional predictive power). In a more particular example, if training data 302 includes 320 patients, those 320 patients can be associated with a root node, and can be divided by determining a feature (e.g., a clinical variable, or a biomarker level) along which to split the group. If a feature is categorical (e.g., sex, hormonal excess, mode of discovery), the group can be divided based on category membership, whereas if a feature is continuous, the feature can be discretized prior to building the tree and/or model (e.g., age can be discretized into multiple binary features, e.g., <20, <30, etc.), and a single discretized feature can be used to split the group associated with the root node. While a single tree could provide some predictive power, decision trees are considered weak learners and alone provide limited accuracy, performance is typically heavily biased by the data that the decision tree is trained on. Note that in some embodiments, an initial tree (e.g., first tree 306) can be a decision tree that is trained using the actual diagnostic classes. However, a first tree can also be generated using a constant that minimizes error (i.e., the observed diagnoses y used for training can all be set to the same value, such as benign, which is closest to an average diagnosis).

In some embodiments, the accuracy of a final trained model can be increased using any suitable technique or combination of techniques. For example, GBM techniques can be used to increase the predictive power of first tree 306 by iteratively adding additional trees that each reduce the error when added to all of the previous trees. In such embodiments, the predictions made by the first tree 306 for each patient can be used to generate a first set of residuals 308 that represent the error in the prediction. In some embodiments, the error can be generated using any suitable loss function, which can be used to generate pseudo-residual values and first residuals 308 can be the pseudo-residuals. For example, a multinomial likelihood loss function can be utilized, which can account for the three possible adrenal mass classes. In such an example, for each patient, a predicted probability of each of the 3 classes can be estimated with the constraint that the predictions must sum up to 1 (i.e., the classes are mutually exclusive and exhaustive). The multinomial likelihood loss function for an individual patient can then be the natural log of the predicted probability for the labeled class associated with that patient, such that the loss function equals 0 if the patient is correctly predicted to have their true class with probability 1 (i.e. ln(1)=0). The expected multinomial likelihood loss function can then be calculated as the average loss estimate across all patients in the dataset.

In some embodiments, first residuals 308 can then be used to train a second tree 310, which can be used to generate second residuals, and so on, until a set of (m−1)^{th} residuals 312 are used to train a final M^{th} tree 314. In some embodiments, the number of trees m used to generate a final model is a hyperparameter that can be set at a particular number or determined based on whether generating an additional tree (e.g., an additional decision tree) would improve the performance of the overall model.

In some embodiments, a trained model 320 can be an aggregation of all of the individual trees 306, 310, . . . , 314, and a trained model can be generated for each unique combination of folds (e.g., models 1-k can be generated with a k^{th} model 322 generated based on the k^{th} set of labeled data). In some embodiments, test data 304 that was reserved from each combination of training data can be used to evaluate the performance of each of the trained models (e.g., first trained model 320 can be evaluated based on the fold reserved from training data 302, while k^{th} model 322 can be evaluated based on the fold reserved from k^{th} training data). In some embodiments, first trained model 320 generates a set of predictions 332 using the test data 304, k^{th} model 322 generates a set of predictions 334 using the k^{th} test data, and each other model is used to make a similar set of predictions based on corresponding test data that was not used during the training process.

In some embodiments, the performance of each model can be calculated based on a comparison of the predictions (e.g., predictions 332 to 334) to the labels associated with the corresponding test data (e.g., based on test data 304, etc.), to generate performance metrics 342 to 344 corresponding to each of the k models. Additionally, in some embodiments, each combination of training data and test data can be used to generate multiple models with various hyperparameters in a grid search operation. For example, the same combination of training data (e.g., training data 302) and test data (e.g., test data 304) can be used to generate multiple different trained models 320 to 322 using different combinations of hyperparameters. In a more particular example, for each set of hyperparameters in the search space that is selected, a k-fold cross validation process can be used to determine performance characteristics associated with the set of hyperparameters. A set of hyperparameters that has the most desirable performance characteristics can be used to training the final model. In some embodiments, the search space can include any suitable range of maximum interactions depth, learning rate (sometimes referred to as shrinkage), and number of trees. For example, the search space can include interaction depths of 1, 2, and 3. As another example, the search space can include a learning rate in a range of 0.01 to 0.001. As yet another example, the search space can include a number of tress in a range of 100 to 5000.

In some embodiments, a final trained model 324 can be generating using hyperparameters that generated the best performance (e.g., where best can be determined using various different metrics). For example, after determining a set of hyperparameters that generate a desired performance, a new GBM of decision trees can be generated using all of the data (i.e., all k folds of data, rather than k-1 folds for training with one fold withheld for testing) and the final set of hyperparameters.

Alternatively, in some embodiments, final trained model 324 can be based on one or more of the trained models (e.g., models 320 to 322). For example, in some embodiments, the model that minimized one or more undesirable metrics (e.g., false negatives, false positives, etc.) or maximized one or more desirable metrics (e.g., specificity, true positives, true negatives, etc.) can be selected as a best performing model and used as final trained model 324. As another example, the performance of each of the k models can be evaluated, and the models can be combined to generate final model 324. In a more particular example, each trained model 320 to 322 can be assigned a weight based on the performance associated with that model (e.g., performance 342 to 344 respectively), and a final output of final trained model 324 can be based on a weighted combination of each of the k trained models.

In some embodiments, after training is complete, unlabeled data 352 corresponding to a patient having an undiagnosed adrenal mass can be provided as input to final trained model 324, and final trained model 324 can provide a prediction 354 of a classification of the adrenal mass.

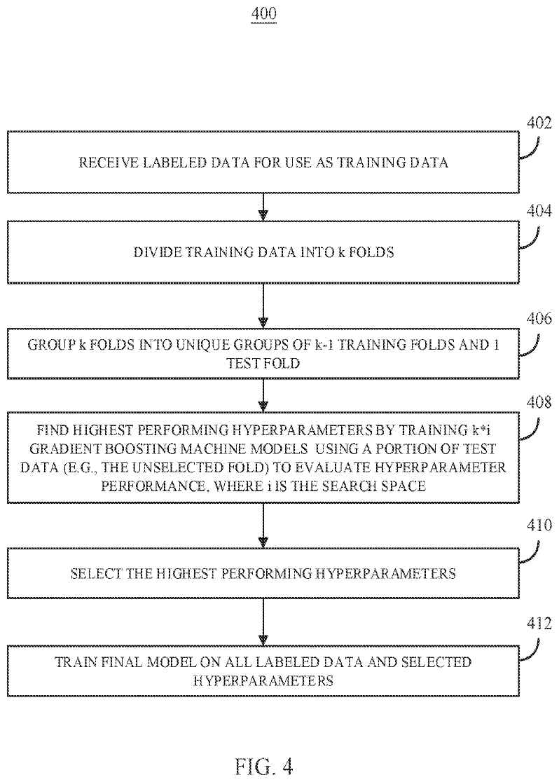

FIG. 4 shows an example 400 of a process for training a machine learning model for automatically predicting a classification of incidental adrenal tumors based on clinical variables and urinary steroid levels in accordance with some embodiments of the disclosed subject matter. As shown in FIG. 4, at 402, process 400 can receive labeled data for use as training data. As described above, process 400 can receive the labeled data from any suitable source, and the training data can include data related to any suitable variables, such as clinical variables and/or biomarkers.

At 404, process 400 can divide the labeled data into k folds that each have a similar distribution of diagnoses to the overall distribution. In some embodiments, any suitable technique or combination of techniques can be used to divide the labeled training data, such as by randomly assigning patients with each diagnoses across the k folds.

At 406, process 400 can generate groupings of the folds into unique combinations of k-1 folds as training data and 1 fold as validation and/or testing data, such that each fold is used as a test fold with the k other folds as training folds.

At 408, process 400 can find a set of highest performing hyperparameters by training k*i decision tree-based GBMs, each having different hyperparameters, where i is a search space of the hyperparameters. As described above in connection with FIG. 3, the performance of each model can be measured during and/or after training to determine which hyperparameters produce the highest performing models. For example, accuracy, positive predictive value, negative predictive value, and other suitable performance characteristics can be calculated for one or more thresholds. In a more particular example, such performance characteristics can be calculated for naïve thresholds (e.g., over 50%). Various metrics (e.g., Youden's J) can be calculated at different cutoff thresholds using the evaluation subset, and the results can be used to calculate performance metrics (e.g., based on a resulting confusion matrix).

In some embodiments, process 400 can perform a search over any suitable hyperparameters such as the maximum number of trees (m) allowed, the maximum interaction depth allowed, and learning rate. The number of trees can be used to limit the total number of decision trees included in the model. The interaction depth can be used to limit the number of splits that are allowed in each of the constituent trees, which can control the degree of interactions between predictor variables. For example, an interaction depth of one implies a model that is purely additive, while an interaction depth of two allows for first order interactions. More generally, an interaction depth of n allows interactions up to order n-1. The shrinkage hyperparameter can be used to modify the learning rate of the algorithm as each additional tree is added to the model. As described above, using grid search techniques to select hyperparameters can include trained and evaluated models identically across a wide selection of parameter combinations. Such techniques are generally more computationally intensive than other techniques such as random search or Bayesian optimization, but can account for a greater variety of parameters. However, such other techniques can also be used in lieu of grid search techniques.

While the mechanisms described herein are generally described in connection with a multinomial (specifically, a three-class) target distributions, binomial target distributions can also be used. For example, multiple models can be built which can include a model that makes a benign-vs-malignant prediction, and another model that makes an ACC-vs-other malignancy prediction. In such an example, the output of the different models can be used in connection with one another to predict the specific multinomial classification of a particular adrenal mass.

At 410, process 400 can select the highest performing hyperparameters based on the performance of the models trained at 408 on test data. In some embodiments, performance can be evaluated by comparing Cohen's Kappa for models that make a multinomial (e.g., three-class) prediction, and comparing the area under the receiver operating characteristic curve (AUC) for models that make a binomial (two-class) prediction. The performance can be evaluated based on the predictions made for the out-of-sample cross-validation results. In some embodiments, the hyperparameters for the final model can be selected based on the multinomial model that minimized the false negative rate. This can insure that as few malignant tumors as possible are misclassified as being benign, while still reducing the number of unnecessary procedures that are performed by giving a practitioner high confidence that indeterminate masses classified as benign are unlikely to have been misclassified ACCs or other malignancies.

At 412, process 400 can train a final model using all of the labeled data and the hyperparameters selected at 410. For example, process 400 can train a decision tree-based GBM with a multinomial classifier using the hyperparameters selected at 410. Other than using all of the data (e.g., not withholding a test set), training of the final model can be performed using techniques described above for training models used to evaluate various hyperparameters.

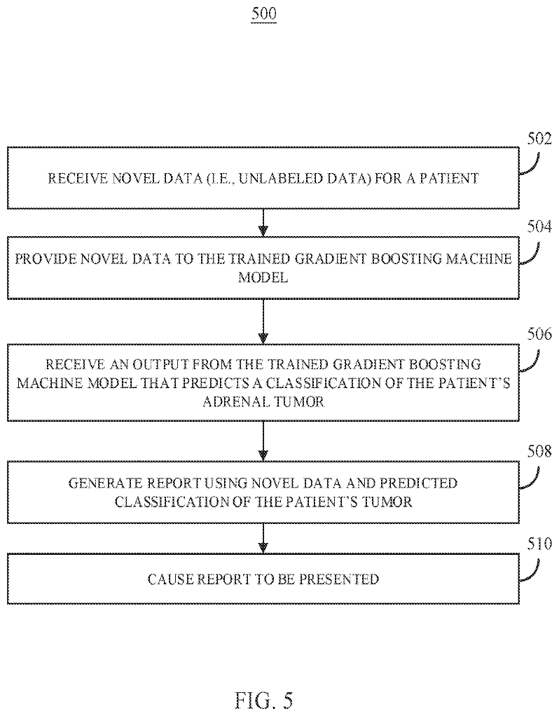

FIG. 5 shows an example 500 of a process for using a machine learning model for automatically predicting a classification of incidental adrenal tumors based on clinical variables and urinary steroid levels in accordance with some embodiments of the disclosed subject matter. As shown in FIG. 5, process 500 can begin at 502 by receiving novel data associated with a patient having an adrenal mass that has not been definitively diagnosed. For example, process 500 can receive clinical variables and biomarker levels associated with the patient from any suitable source (e.g., data source 102).

At 504, process 500 can provide novel data to a trained GBM model in a format that matches a format of the training data. For example, process 500 can provide the novel data to a final GBM model trained at 412, or final trained model 324.

At 506, process 500 can receive an output from the trained GBM model that is a prediction of a classification of the patient's adrenal tumor. In some embodiments, the output can be in any suitable format. For example, the output can be in a format that provides a likelihood that the adrenal mass is each of three classes of mass (e.g., benign, ACC, and other malignancy).

At 508, process 500 can generate a report using the novel data and the predicted classification of the patient's tumor. In some embodiments, the report can include any suitable information and can be in any suitable format.

At 510, process 500 can cause the report to be presented to a user. For example, process 500 can cause the report to be presented to a physician treating the patient (e.g., using computing device 110) in response to a request from the physician and/or in response to the physician accessing an electronic medical record associated with the patient.

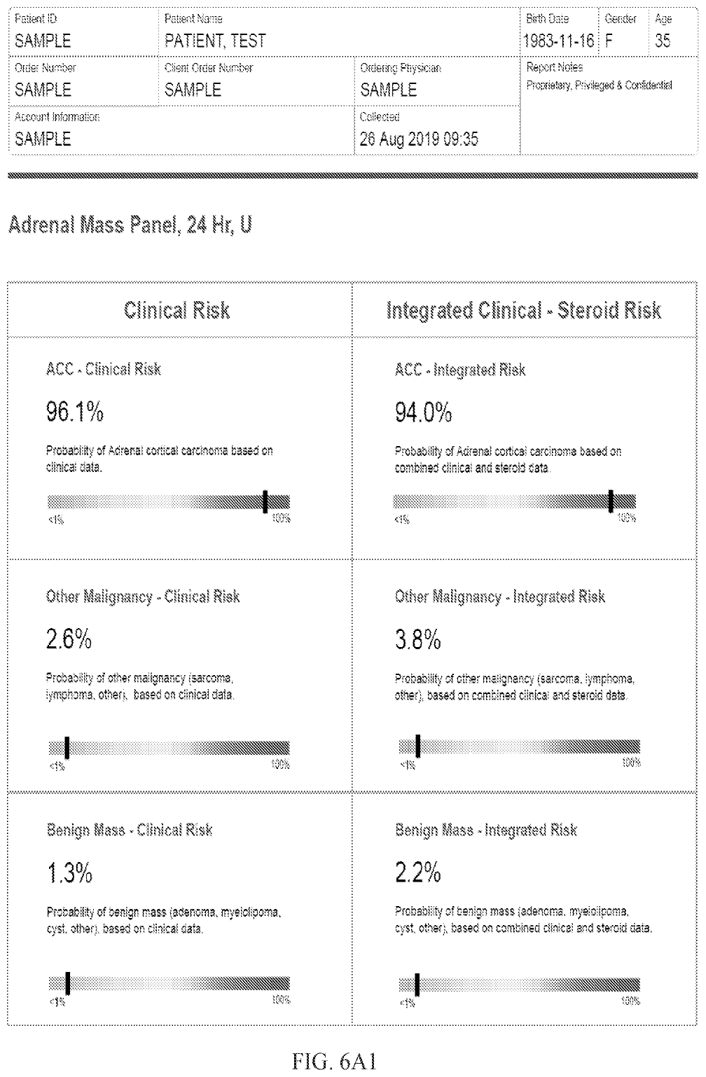

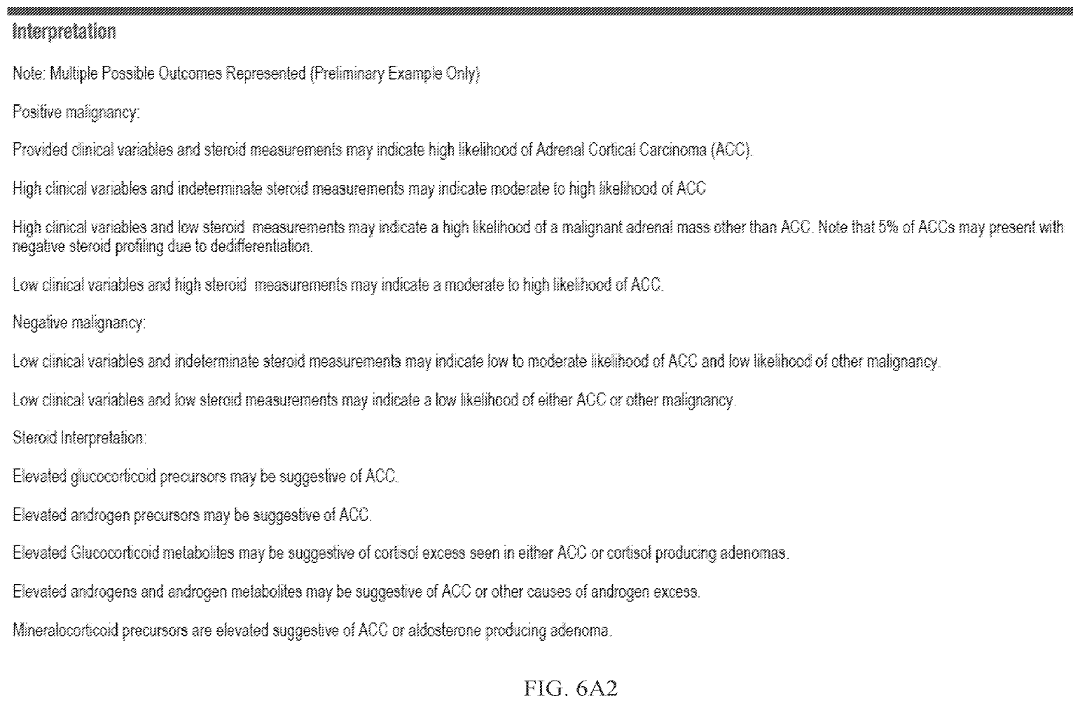

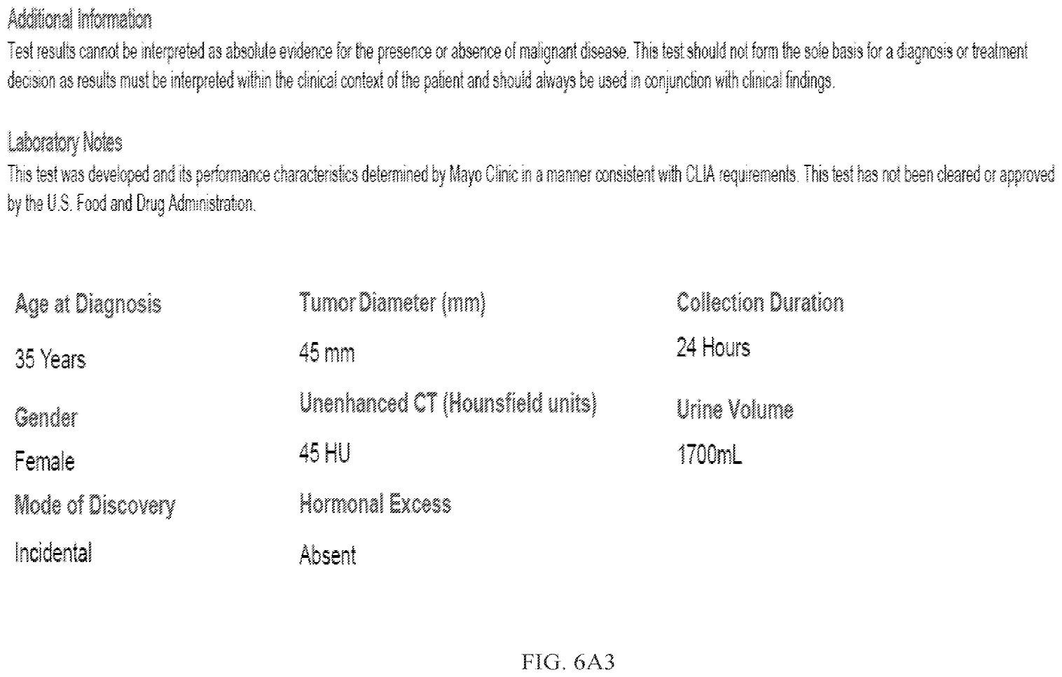

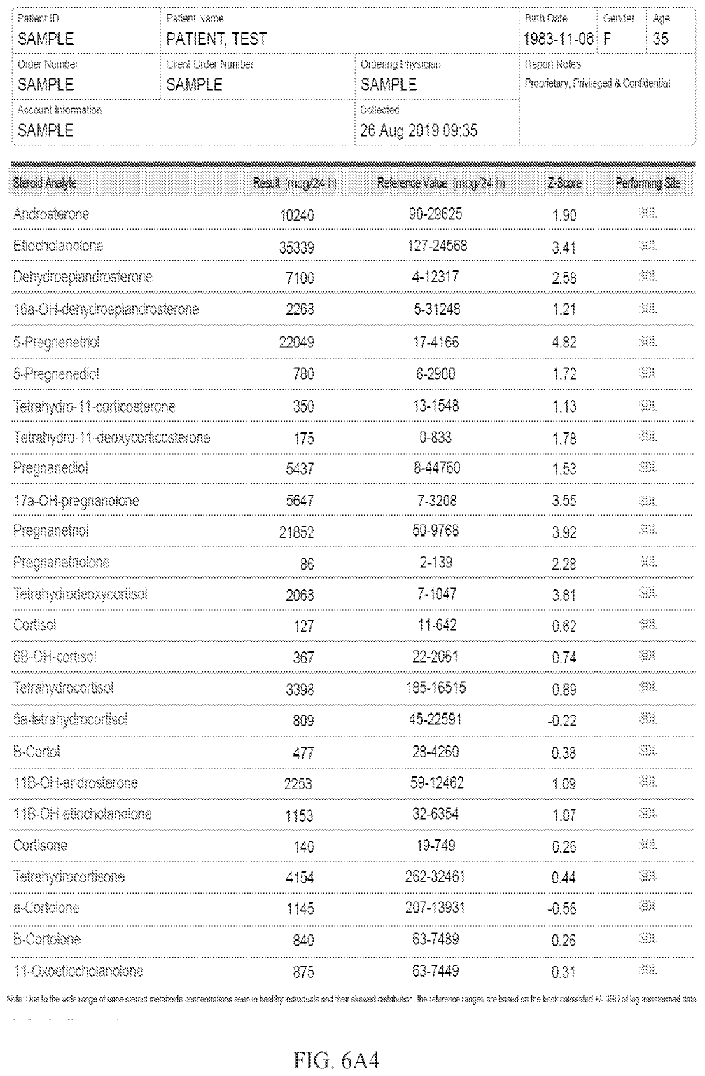

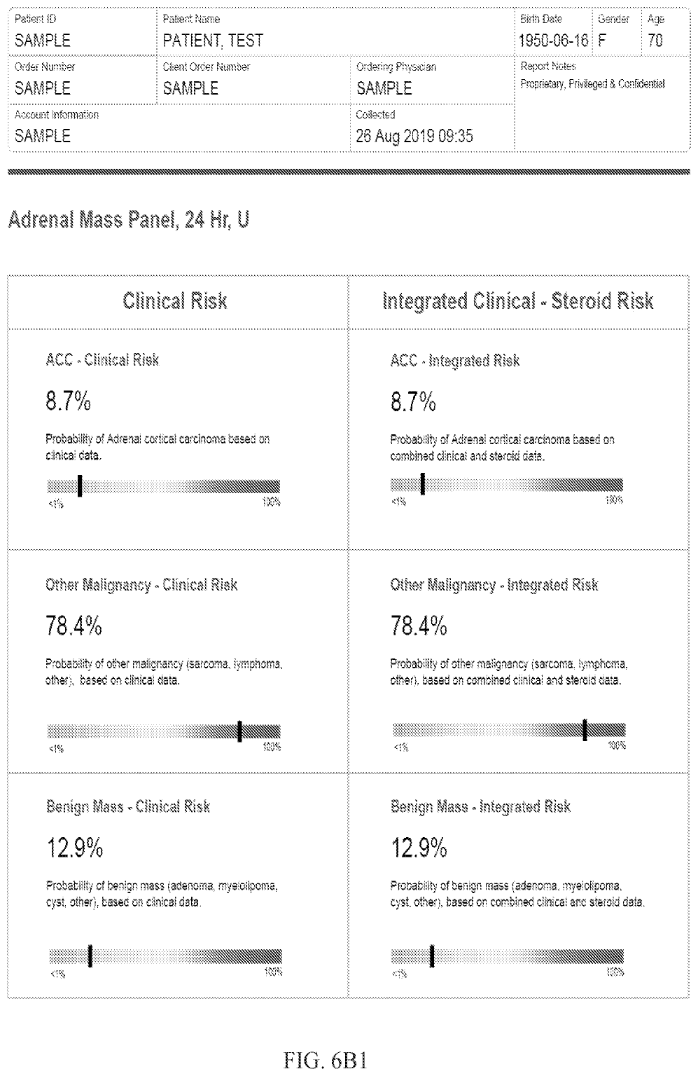

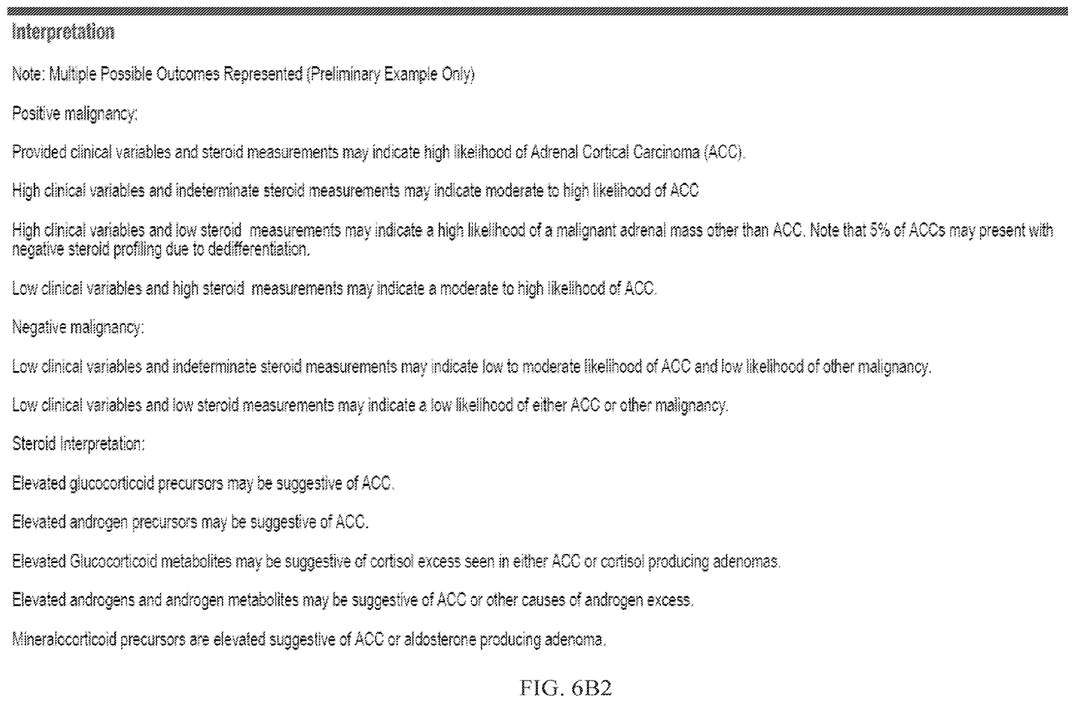

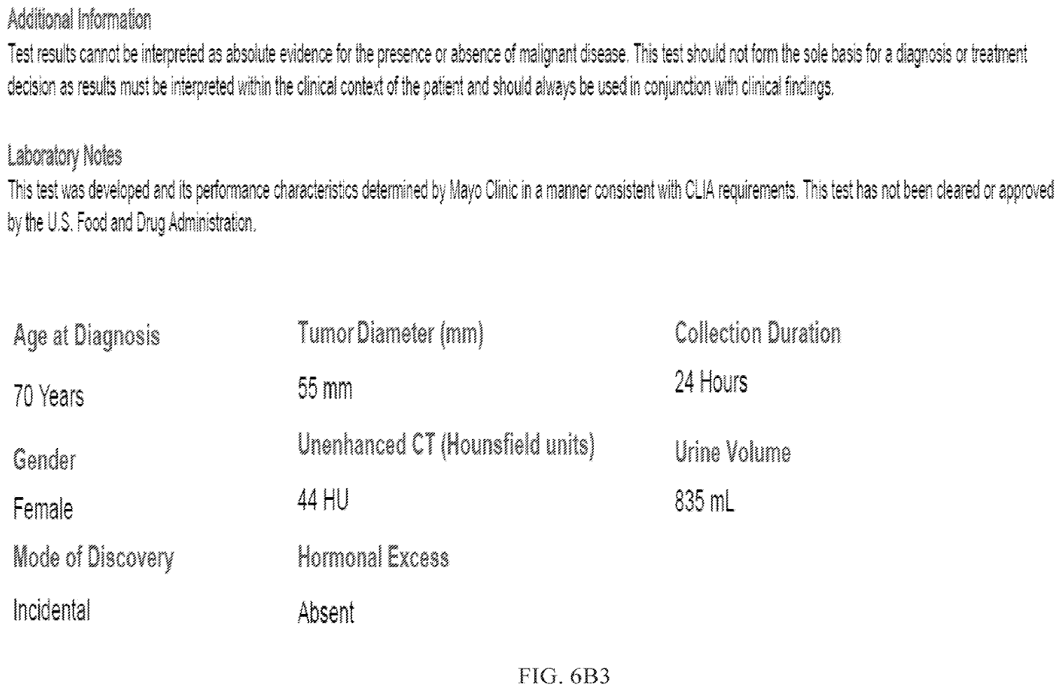

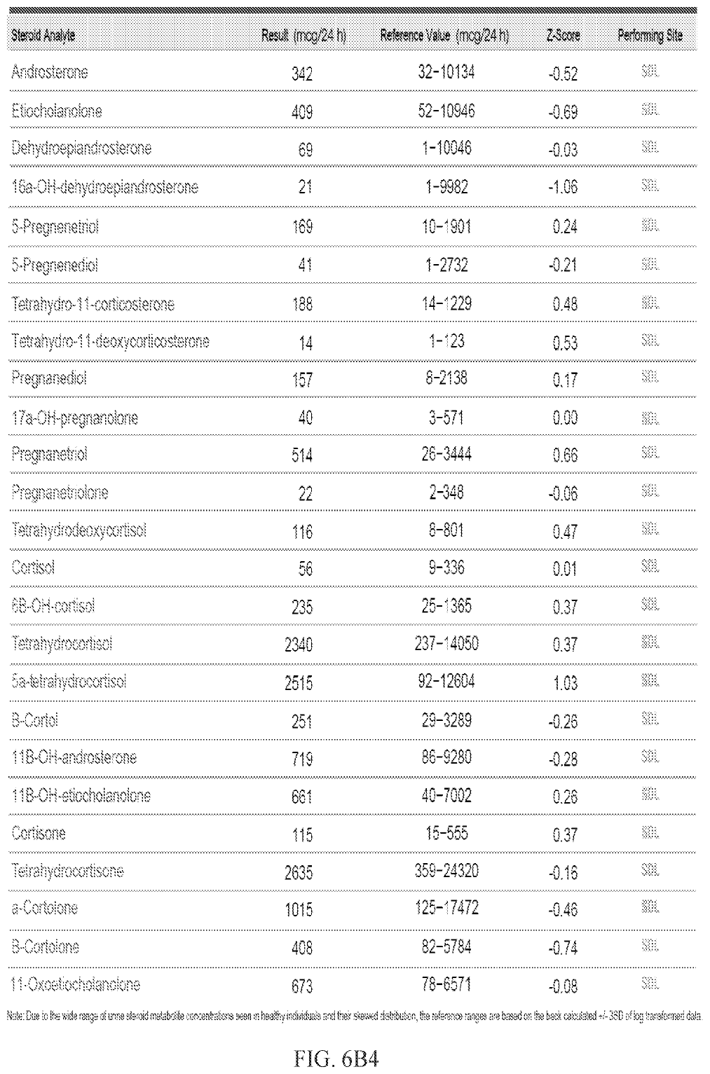

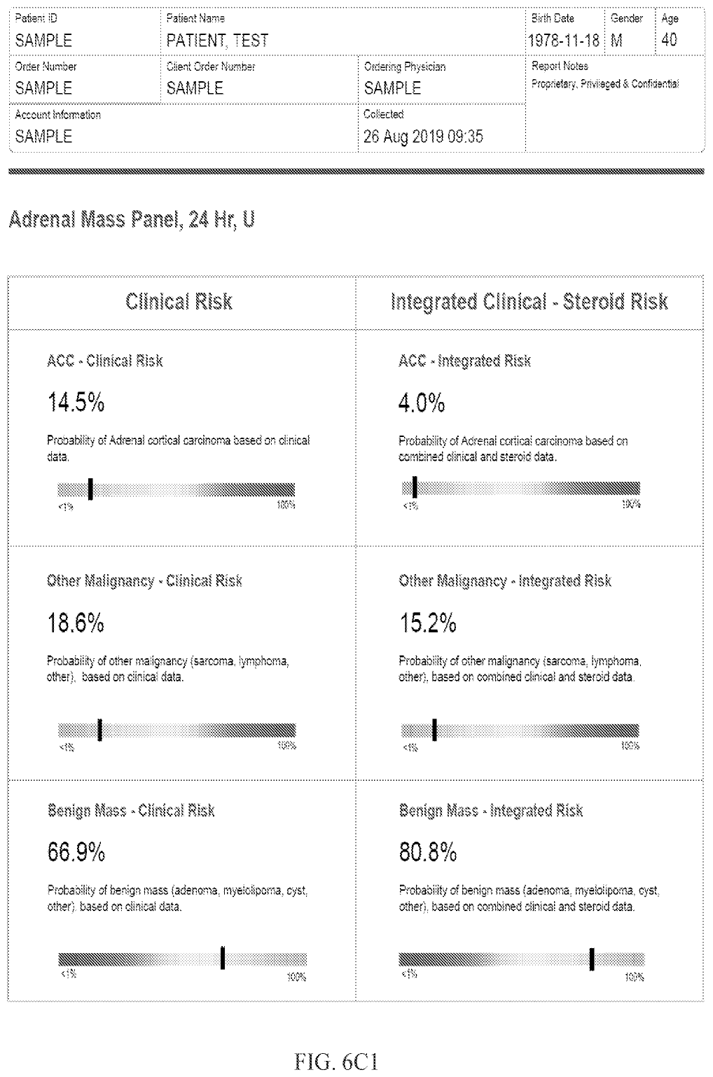

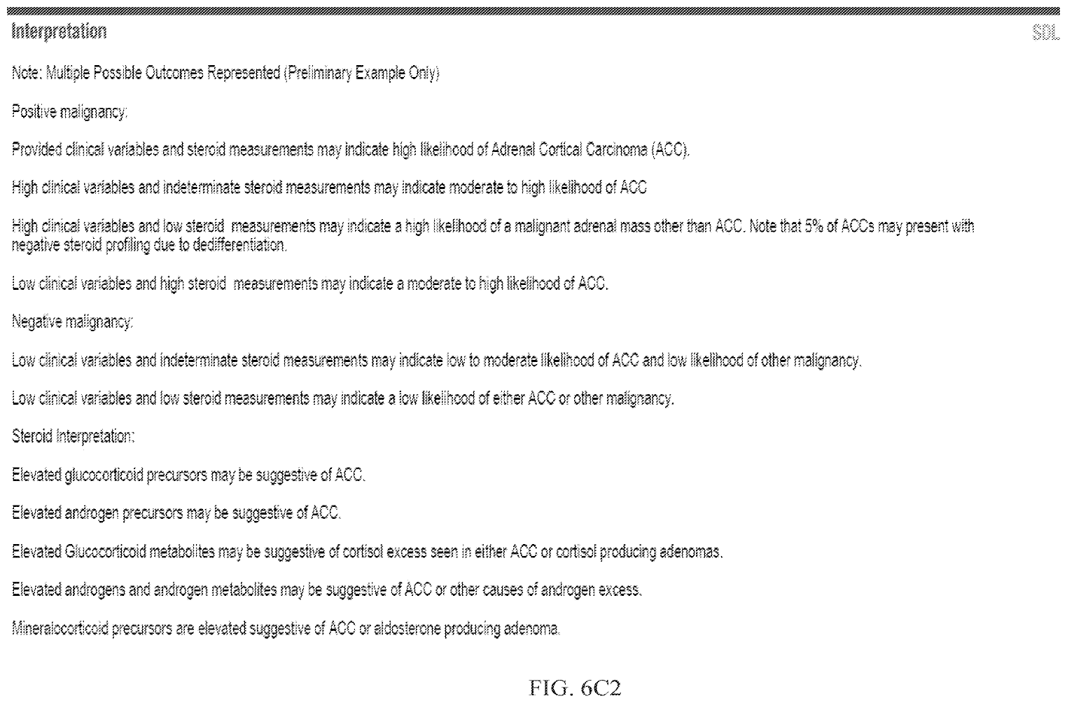

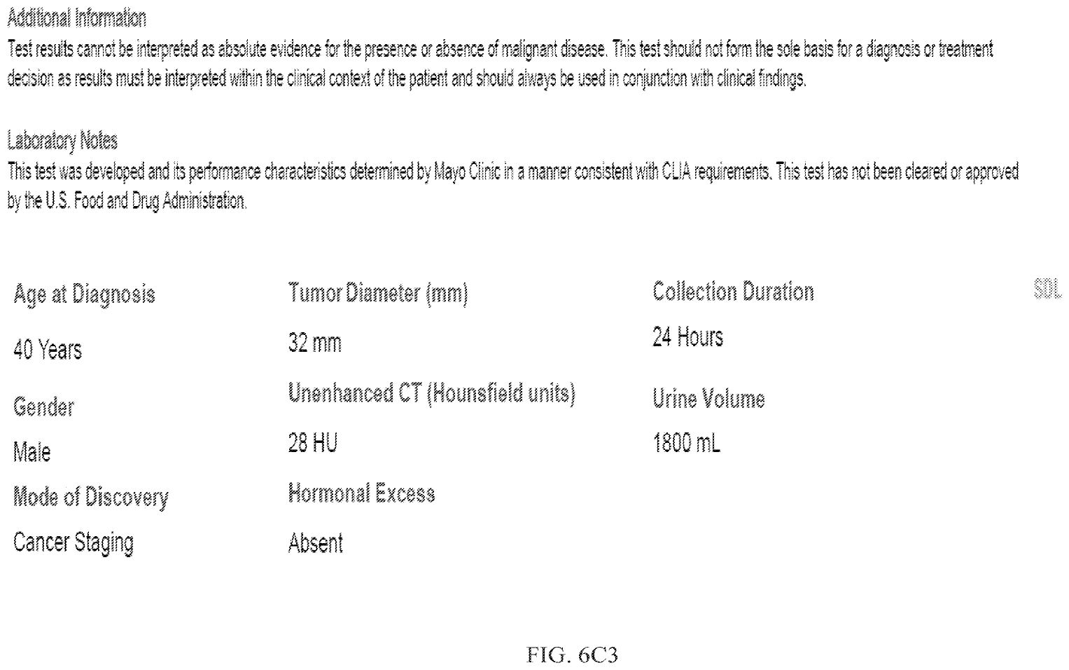

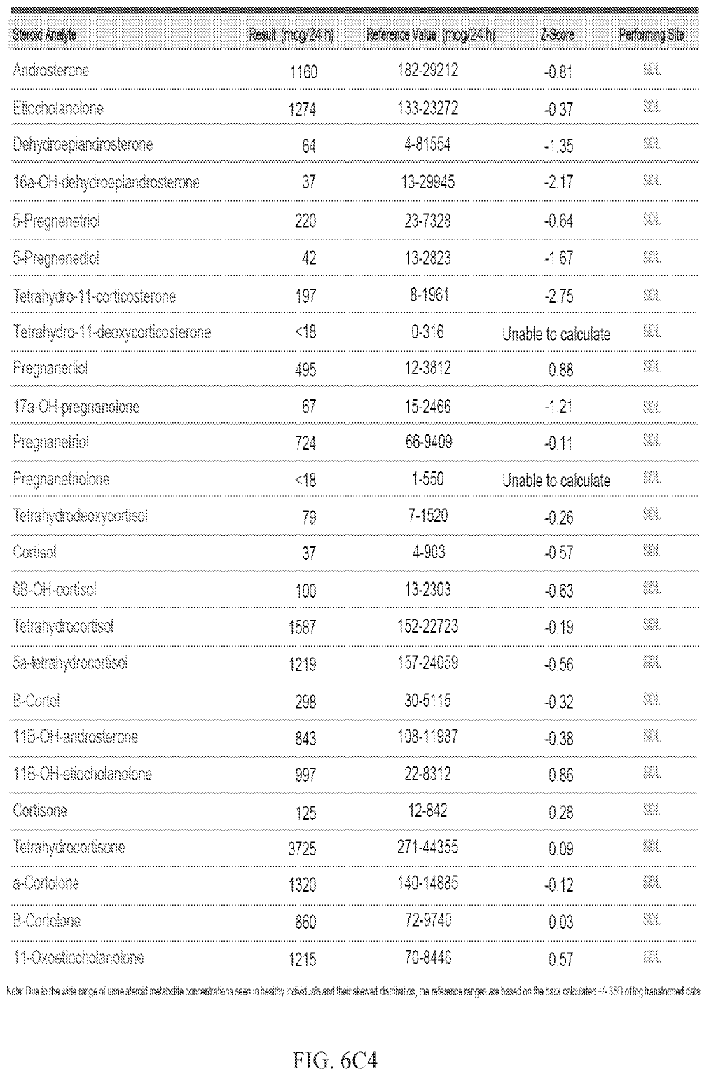

FIGS. 6A1 to 6A4 show an example of a report that can be generated based on an output of a system for automatically predicting a classification of incidental adrenal tumors based on clinical variables and urinary steroid levels in accordance with some embodiments of the disclosed subject matter. As shown in FIG. 6A1, the report can include a likelihood that an adrenal mass belongs to each class that was generated by a trained GBM model (e.g., the final GBM model trained at 412, or final trained model 324). In some cases, a prediction based on only the clinical variables can also be presented. For example, a prediction based on clinical parameters only can be determined and presented prior to steroid profiling, and can be used to determine whether steroid profiling is called for. In a particular example, if a prediction based on clinical variables has a 90-100% prediction for a benign lesion, proceeding with steroid profiling/integrated prediction may not be needed and the cost associated with steroid profiling can be avoided. The two predictions can be shown together, as shown in FIG. 6A1, to provide information about how the prediction(s) has changed based on the addition of steroid profiling data. As shown in FIG. 6A2, the report can include guidance for interpreting the results to facilitate a physician making a more informed diagnosis that is not solely reliant on the machine learning model. As shown in FIG. 6A3, the report can include the relevant clinical information that was used to make the predictions shown in FIG. 6A1, including age at diagnosis, tumor diameter, sex, mode of discovery, the unenhanced Hounsfield units of the tumor from a CT, and the presence or absence of hormonal excess. FIG. 6A3 also includes information about the urine test that was used to determine steroid levels, including collection duration and volume. As shown in FIG. 6A4, the levels of the various steroids measured from the patient's urine sample can be included in the report. The results can be presented as a raw level (e.g., in micrograms per 24 hours), and a reference value (based on control ranges derived from patients without an adrenal mass) can also be presented to assist in interpretation. The report can also include a z-score associated with each of the steroids (an indication of how far from the mean the value is). In some embodiments, a z-score greater than 3 can be considered abnormal and can be highlighted on a graphical user interface (not shown).

FIGS. 6B1 to 6B4 show another example of a report that can be generated based on an output of a system for automatically predicting a classification of incidental adrenal tumors based on clinical variables and urinary steroid levels in accordance with some embodiments of the disclosed subject matter.

FIGS. 6C1 to 6C4 show yet another example of a report that can be generated based on an output of a system for automatically predicting a classification of incidental adrenal tumors based on clinical variables and urinary steroid levels in accordance with some embodiments of the disclosed subject matter.

Mechanisms described herein were used to generate trained models based on only steroid data, and based on clinical and steroid data. Table 1 shows the performance (as a confusion matrix) of the model based on only steroid data, and Table 2 shows the performance (as a confusion matrix) of the model based on both the clinical and steroid data. The results are based on performance of models trained during cross-validation on the test data.

TABLE 1
Steroid Only Model - Confusion Matrix and Statistics

| | | Reference | | |
|---|---|---|---|---|
| | | Benign | ACC | Other Mal. |
| Predicition | Benign | 350 | 5 | 21 |
| | ACC | 0 | 24 | 0 |
| | Other Mal. | 1 | 0 | 0 |

TABLE 2
Steroid + Clinical Model - Confusion Matrix and Statistics

| | | Reference | | |
|---|---|---|---|---|
| | | Benign | ACC | Other Mal. |
| Prediction | Benign | 348 | 3 | 12 |
| | ACC | 0 | 25 | 1 |
| | Other Mal. | 3 | 1 | 8 |

The importance of the different variables for each of the models were calculated based on Friedman's proposal for relative influence, and the importance of the top 20 most important variables is listed in Table 3 for the steroid only model, and in Table 4 for the steroid and clinical model.

TABLE 3
Steroid Only Model - GBM variable importance

| | Overall prediction |
|---|---|
| Variable name | importance |
| ‘_5_PT’ | 100.000 |
| THS | 73.417 |
| ‘_5_PD’ | 56.326 |
| ‘_16a_DHEA’ | 47.746 |
| ‘_6b_OH_Cortisol’ | 23.990 |
| THB | 20.139 |
| THE | 18.140 |
| ‘_11b_OH_Etio’ | 17.333 |
| ANDROS | 16.265 |
| a_cortolone | 13.382 |
| ‘_5a_THF’ | 11.137 |
| ‘_11_oxo_Etio’ | 9.702 |
| PT | 9.398 |
| ‘_17_HP’ | 8.983 |
| PD | 8.869 |
| THF | 8.639 |
| Cortisol | 8.397 |
| ‘_11b_OH_Andro’ | 6.506 |
| Cortisone | 5.998 |
| TH-DOC | 5.692 |

TABLE 4
Steroid + Clinical Model - GBM variable importance

| | Overall prediction |
|---|---|
| Variable name | importance |
| Hounsfield units | 100.000 |
| THS | 66.242 |
| ‘_5_PT’ | 56.947 |
| Size | 43.975 |
| hormoneTRUE | 20.783 |
| ‘_5_PD’ | 18.528 |
| ‘_11b_OH_Etio’ | 10.204 |
| mode of disc. | 7.477 |
| PD | 6.571 |
| TH_DOC | 5.448 |
| DHEA | 5.016 |
| Cortisol | 4.970 |
| maleTRUE | 4.532 |
| ‘_16a_DHEA’ | 4.310 |
| PT | 4.036 |
| Cortisone | 3.895 |
| ANDROS | 3.516 |
| THF | 3.100 |
| ‘_6b_OH_Cortisol’ | 2.973 |
| ‘_17_HP’ | 1.688 |

Appendix A, Appendix B, and Appendix C filed in U.S. Provisional Application No. 62/944,140 include explanations and examples related to the disclosed subject matter, and each is hereby incorporated by reference herein in its entirety.

### Further Examples Having a Variety of Features

Example 1: A method for predicting a classification of an adrenal mass, the method comprising: generating a feature vector that includes a first plurality of values and a second plurality of values, wherein the first plurality of values corresponds to a respective plurality of clinical variables associated with a patient presenting with an unclassified adrenal mass, and the second plurality of values corresponds to a respective plurality of biomarker levels associated with the patient presenting with the unclassified adrenal mass; providing the feature vector to a trained machine learning model, wherein the machine learning model was trained using a plurality of labeled feature vectors associated with a respective plurality of patients having a classified adrenal mass, wherein each of the plurality of feature vectors included values corresponding to the plurality of clinical variables and the plurality of biomarker levels associated with a respective patient, and each of the plurality of feature vectors is associated with an indication of a diagnosis of the respective classified adrenal mass as being one of benign, adrenal cortical carcinoma (ACC), and a malignant adrenal mass other than ACC; receiving, from the trained machine learning model, an output indicative of a classification of the unclassified adrenal mass; and causing information indicative of the classification to be presented to a user to aid the user in classification of the unclassified adrenal mass.

Example 2: A method for predicting a classification of an adrenal mass, the method comprising: generating a feature vector that includes a first plurality of values and a second plurality of values, wherein the first plurality of values corresponds to a respective plurality of clinical variables associated with a patient presenting with an unclassified adrenal mass, and the second plurality of values corresponds to a respective plurality of biomarker levels associated with the patient presenting with the unclassified adrenal mass; providing the feature vector to a trained machine learning model; receiving, from the trained machine learning model, an output indicative of a classification of the unclassified adrenal mass; and causing information indicative of the classification to be presented to a user to aid the user in classification of the unclassified adrenal mass.

Example 3: The method of Example 2, wherein the machine learning model was trained using a plurality of labeled feature vectors associated with a respective plurality of patients having a classified adrenal mass.

Example 4: The method of any one of Examples 2 or 3, wherein each of the plurality of feature vectors included values corresponding to the plurality of clinical variables and the plurality of biomarker levels associated with a respective patient.

Example 5: The method of any one of examples 2 to 4, wherein each of the plurality of feature vectors is associated with an indication of a diagnosis of the respective classified adrenal mass as being one of benign, adrenal cortical carcinoma (ACC), and a malignant adrenal mass other than ACC

Example 6: The method of any one of examples 1 to 5, wherein the trained machine learning model is a gradient boosting machine model comprising a plurality of decision trees.

Example 7: The method of any one of examples 1 to 6, wherein the plurality of clinical variables includes an unenhanced Hounsfield unit value of the adrenal mass, a size of the adrenal mass, and an indication of whether the patient was experiencing an excess of hormones excreted by the adrenal gland.

Example 8: The method of any one of examples 1 to 7, wherein the plurality of biomarker levels includes at least ten levels of biomarkers indicative of at least one of a steroid, a steroid precursor, and a metabolite that falls within the mineralocorticoid, glucocorticoid, or androgen pathways of adrenal steroidogenesis extracted from a 24-hour urine sample.

Example 9: The method of any one of examples 1 to 8, wherein the output comprises a plurality of values each indicative of a likelihood that the unclassified adrenal mass is a member of each class of adrenal mass, wherein the classes of adrenal mass comprise benign, ACC, and malignant adrenal mass other than ACC.

Example 10: The method of any one of examples 1 to 9, further comprising: receiving a plurality of biomarker levels from a liquid chromatography high-resolution accurate-mass (LC-HRAM) spectrometer; and generating the second plurality of values using the plurality of biomarker levels.

Example 11: The method of any one of examples 1 to 10, wherein the second plurality of values comprises a plurality of z-scores each indicative of a level of a particular biomarker.

Example 12: The method of any one of examples 1 to 11, further comprising: receive the plurality of clinical variables from an electronic medical record system; and generate the first plurality of values using the plurality of clinical variables.

Example 13: A system comprising: at least one hardware processor that is configured to: perform a method of any one of Examples 1 to 12.

Example 14: A non-transitory computer readable medium containing computer executable instructions that, when executed by a processor, cause the processor to perform a method of any one of Examples 1 to 12.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above-described steps of the processes of FIGS. 4 and 5 can be executed or performed in any order or sequence not limited to the order and sequence shown and described in the figures. Also, some of the above steps of the processes of FIGS. 4 and 5 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. A system for predicting a classification of an adrenal mass, the system comprising:
at least one hardware processor that is programmed to:
generate a feature vector that includes a first plurality of values and a second plurality of values,
wherein the first plurality of values corresponds to a respective plurality of clinical variables associated with a patient presenting with an unclassified adrenal mass, and
the second plurality of values corresponds to a respective plurality of biomarker levels associated with the patient presenting with the unclassified adrenal mass;
provide the feature vector to a trained machine learning model, wherein the machine learning model was trained using a plurality of labeled feature vectors associated with a respective plurality of patients having a classified adrenal mass,
wherein each of the plurality of feature vectors included values corresponding to the plurality of clinical variables and the plurality of biomarker levels associated with a respective patient, and
each of the plurality of feature vectors is associated with an indication of a diagnosis of the respective classified adrenal mass as being one of benign, adrenal cortical carcinoma (ACC), and a malignant adrenal mass other than ACC;
receive, from the trained machine learning model, an output indicative of a classification of the unclassified adrenal mass; and
cause information indicative of the classification to be presented to a user to aid the user in classification of the unclassified adrenal mass.

2. The system of claim 1, wherein the trained machine learning model is a gradient boosting machine model comprising a plurality of decision trees.

3. The system of claim 1, wherein the plurality of clinical variables includes an unenhanced Hounsfield unit value of the adrenal mass, a size of the adrenal mass, and an indication of whether the patient was experiencing an excess of hormones excreted by the adrenal gland.

4. The system of claim 1, wherein the plurality of biomarker levels includes at least ten levels of biomarkers indicative of at least one of a steroid, a steroid precursor, and a metabolite that falls within the mineralocorticoid, glucocorticoid, or androgen pathways of adrenal steroidogenesis extracted from a 24-hour urine sample.

5. The system of claim 1, wherein the output comprises a plurality of values each indicative of a likelihood that the unclassified adrenal mass is a member of each class of adrenal mass, wherein the classes of adrenal mass comprise benign, ACC, and malignant adrenal mass other than ACC.

6. The system of claim 1, further comprising a liquid chromatography high-resolution accurate-mass (LC-HRAM) spectrometer, and
wherein the at least one hardware processor that is further programmed to:
receive a plurality of biomarker levels from the LC-HRAM spectrometer; and
generate the second plurality of values using the plurality of biomarker levels.

7. The system of claim 1, wherein the second plurality of values comprises a plurality of z-scores each indicative of a level of a particular biomarker.

8. The system of claim 1, wherein the at least one hardware processor that is further programmed to:
receive the plurality of clinical variables from an electronic medical record system; and
generate the first plurality of values using the plurality of clinical variables.

9. A method for predicting a classification of an adrenal mass, the method comprising:
generating a feature vector that includes a first plurality of values and a second plurality of values,
wherein the first plurality of values corresponds to a respective plurality of clinical variables associated with a patient presenting with an unclassified adrenal mass, and
the second plurality of values corresponds to a respective plurality of biomarker levels associated with the patient presenting with the unclassified adrenal mass;
providing the feature vector to a trained machine learning model, wherein the machine learning model was trained using a plurality of labeled feature vectors associated with a respective plurality of patients having a classified adrenal mass,
wherein each of the plurality of feature vectors included values corresponding to the plurality of clinical variables and the plurality of biomarker levels associated with a respective patient, and
each of the plurality of feature vectors is associated with an indication of a diagnosis of the respective classified adrenal mass as being one of benign, adrenal cortical carcinoma (ACC), and a malignant adrenal mass other than ACC;
receiving, from the trained machine learning model, an output indicative of a classification of the unclassified adrenal mass; and
causing information indicative of the classification to be presented to a user to aid the user in classification of the unclassified adrenal mass.

10. The method of claim 9, wherein the trained machine learning model is a gradient boosting machine model comprising a plurality of decision trees.

11. The method of claim 9, wherein the plurality of clinical variables includes an unenhanced Hounsfield unit value of the adrenal mass, a size of the adrenal mass, and an indication of whether the patient was experiencing an excess of hormones excreted by the adrenal gland.

12. The method of claim 9, wherein the plurality of biomarker levels includes at least ten levels of biomarkers indicative of at least one of a steroid, a steroid precursor, and a metabolite that falls within the mineralocorticoid, glucocorticoid, or androgen pathways of adrenal steroidogenesis extracted from a 24-hour urine sample.

13. The method of claim 9, wherein the output comprises a plurality of values each indicative of a likelihood that the unclassified adrenal mass is a member of each class of adrenal mass, wherein the classes of adrenal mass comprise benign, ACC, and malignant adrenal mass other than ACC.

14. The method of claim 9, further comprising:
receiving a plurality of biomarker levels from a liquid chromatography high-resolution accurate-mass (LC-HRAM) spectrometer; and
generating the second plurality of values using the plurality of biomarker levels.

15. The method of claim 9, wherein the second plurality of values comprises a plurality of z-scores each indicative of a level of a particular biomarker.

16. The method of claim 9, further comprising:
receive the plurality of clinical variables from an electronic medical record system; and
generate the first plurality of values using the plurality of clinical variables.

17. A non-transitory computer readable medium containing computer executable instructions that, when executed by a processor, cause the processor to perform a method for predicting a classification of an adrenal mass, the method comprising:
generating a feature vector that includes a first plurality of values and a second plurality of values,
wherein the first plurality of values corresponds to a respective plurality of clinical variables associated with a patient presenting with an unclassified adrenal mass, and
the second plurality of values corresponds to a respective plurality of biomarker levels associated with the patient presenting with the unclassified adrenal mass;
providing the feature vector to a trained machine learning model, wherein the machine learning model was trained using a plurality of labeled feature vectors associated with a respective plurality of patients having a classified adrenal mass,
wherein each of the plurality of feature vectors included values corresponding to the plurality of clinical variables and the plurality of biomarker levels associated with a respective patient, and
each of the plurality of feature vectors is associated with an indication of a diagnosis of the respective classified adrenal mass as being one of benign, adrenal cortical carcinoma (ACC), and a malignant adrenal mass other than ACC;
receiving, from the trained machine learning model, an output indicative of a classification of the unclassified adrenal mass; and
causing information indicative of the classification to be presented to a user to aid the user in classification of the unclassified adrenal mass.

18. The non-transitory computer readable medium of claim 17, wherein the trained machine learning model is a gradient boosting machine model comprising a plurality of decision trees.

19. The non-transitory computer readable medium of claim 17, wherein the plurality of clinical variables includes a an unenhanced Hounsfield unit value of the adrenal mass, a size of the adrenal mass, and an indication of whether the patient was experiencing an excess of hormones excreted by the adrenal gland.

20. The non-transitory computer readable medium of claim 17, wherein the plurality of biomarker levels includes at least ten levels of biomarkers indicative of at least one of a steroid, a steroid precursor, and a metabolite that falls within the mineralocorticoid, glucocorticoid, or androgen pathways of adrenal steroidogenesis extracted from a 24-hour urine sample.

21-24. (canceled)
